(19) United States
(12) Patent Application Publication
(10) Pub. No.: US 2012/0183473 A1
(43) Pub. Date: Jul. 19, 2012

(54) ACTIVE INGREDIENT AND METHOD FOR TESTING AN ACTIVE INGREDIENT

(75) Inventors: Oliver Lade, Berlin (DE); Arno Steckenborn, Berlin (DE)

(73) Assignee: SIEMENS AKTIENGESELLSCHAFT, Muenchen (DE)

(21) Appl. No.: 13/387,915

(22) PCT Filed: Jul. 13, 2010
(86) PCT No.: PCT/EP10/60006

§ 371 Date: Apr. 9, 2012

(30) Foreign Application Priority Data
Jul. 29, 2009 (DE) 10 2009 035 649.5

(51) Int. Cl. A61K 51/10; A61K 51/08; C12Q 1/02; G01N 33/53; C12N 9/00; C07K 16/00

(52) U.S. Cl. 424/1.49; 435/183; 530/387.1; 435/29; 435/7.1; 424/1.69

(57) ABSTRACT

A therapeutically active ingredient is administered to the human or animal body. The active ingredient contains an α-hydroxy acid, an α-amino acid, a peptide or a protein, the component being radiolabeled with ¹¹C and the radioactive atom replacing the C atom of the carboxyl group or of a peptide bond in the a position. The labeled therapeutically active ingredient which is chemically identical to the active ingredient to be used for therapeutic purposes can be advantageously used in tests which allow a dispersion of the active ingredient to be tested in tissue samples or in the human or animal body by positron emission tomography. The complementary information can for example help to accelerate the approval procedure for medicaments.

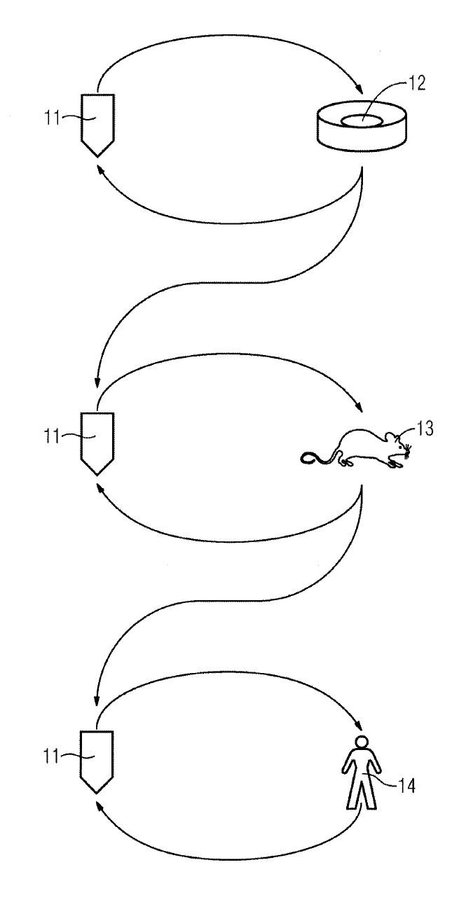

### CROSS REFERENCE TO RELATED APPLICATIONS

This application is based on and hereby claims priority to International Application No. PCT/EP2010/060006 filed on Jul. 13, 2010 and German Application No. 10 2009 035 649.5 filed on Jul. 29, 2009, the contents of which are hereby incorporated by reference.

### BACKGROUND

The invention relates to a therapeutic active ingredient for administration to a human or animal body, which active ingredient comprises one of the following chemical compounds or is formed thereby. The therapeutic active ingredient contains an α-hydroxy acid, an α-amino acid, a peptide or a protein.

Therapeutic active ingredients (also referred to hereinafter as active ingredients for short) are used to a great extent in modern medicine for treating human or animal bodies. For the purposes of this document, therapeutic active ingredients are to be understood to mean substances whose purpose is that of curing diseases or alleviating complaints in the human or animal body. Therapeutic active ingredients are available on the market as drugs, which are a preparation containing the therapeutic active ingredient. Here, use is also made of active ingredients which comprise the chemical constituents listed at the beginning or are formed therefrom. Owing to the complex reactions of the active ingredient in the individual to be treated, comprehensive studies are necessary before introducing active ingredients (drugs) to the market, in order to rule out undesired effects of the active ingredient on the individual to be treated or to reduce them to a tolerable minimum. These experiments are carried out by, inter alia, simulations, animal experiments, and, in the final phase prior to approval of a new drug, also by studying the administration of the drug to humans. In the interests of the test subjects for the studies on the human body, the statements which can be made about the mode of action of the drug are, however, limited in that only a limited number of clinical diagnostic methods are available for studying the living body.

### SUMMARY

It is one possible object to provide therapeutic active ingredients which allow a comparatively extensive study prior to final approval of the active ingredient, and to specify methods for testing therapeutic active ingredients which enable comparatively comprehensive studies to be carried out prior to the approval of a drug.

The inventors propose using the active ingredient mentioned at the beginning and radiolabeling the active ingredient with ¹¹C, wherein the ¹¹C replaces the α-carbon-binding carbon of the carboxyl group of the α-hydroxy acid or of the α-amino acid, or the carbon of the peptide bond of the peptide or of the protein. Thus, the result is that, advantageously, the labeled active ingredient is chemically identical to the active ingredient to be studied, since merely one carbon of the active ingredient is replaced with an ¹¹C, and so, advantageously, the chemical effect of the labeled active ingredient is identical to that of the active ingredient to be studied. Thus, the proposal provides an active ingredient whose effect is predictable owing to prior studies of the identical unlabeled active ingredient, but which, by an examination using positron emission tomography (also referred to hereinafter as PET for short) after administration of the drug, makes it possible to describe the mode of action of the active ingredient to be studied.

Thus, the above-stated object is achieved by the inventors' proposed methods for testing an active ingredient, wherein the active ingredient with radioactive label is used and the distribution of the active ingredient in the test subject is determined by positron emission tomography (also referred to hereinafter as PET for short). Alternatively, it is also possible for the radio labeled active ingredient to be applied to a tissue sample of the tissue to be treated with the active ingredient. Using positron emission tomography, it is then possible to determine the distribution of the active ingredient in the tissue.

Active ingredients or drugs are understood to mean physiologically active agents which bring about a desired change in state during therapeutic treatment of a patient, for example the curing of a disease or the alleviation of complaints. By positron emission tomography, it is possible during testing of the active ingredient to establish how it and its metabolites are distributed in the body. This is because positron emission tomography allows the acquisition of a three-dimensional image of the distribution of the positron-emitting substances in the test subject. The findings obtained in this way during testing of an active ingredient can, advantageously, provide possible information about the mode of action and, in particular, the site of action of active ingredients and the metabolization thereof. For example, it is possible to predict adverse effects which come about as a result of the active ingredient accumulating not only in the organ to be treated, but also in other organs. It is also possible to establish how long an active ingredient takes in order to reach the site of action after intake and how long it acts there.

The method of positron emission tomography is known per se. Typically, this method is carried out as an imaging method for the examination of particular tissues. In the related art, what are known as tracers or biomarkers, which are similar to substances in the body and therefore behave comparably, are prepared for this purpose. As described in the introduction of a dissertation by Kjerstin Bruus-Jensen of the Technical University of Munich from 2006 (“Entwicklung und Evaluierung neuer Methoden zur Radiomarkierung peptidischer Tracer mit ¹⁸F und ^{99M}Tc für die nuklearmedizinische Diagnostik” (“Developing and evaluating new methods for radiolabeling peptide tracers with ¹⁸F und ^{99M}Tc for diagnostic nuclear medicine”)), it is customary to label, for example, peptides with radioactive fluorine. These can be used for an in vivo examination in the human body, and the aim of this diagnostic method is to gain information about pathological states of the examined tissue in the body. It should be noted that the use of ¹⁸F to replace, for example, an OH group in the radiolabeled peptide chemically alters this substance and must take place in a region of the molecule which has a negligible effect on the reaction with the peptide in question in the human body.

In contrast, labeling a therapeutic active ingredient with ¹¹C, as proposed, has the advantage that the labeled substance is chemically identical to the substance to be studied. Thus, the statements which can be made by PET can be applied, without restriction, to the therapeutic active ingredient to be studied.

Advantageously, the therapeutic active ingredient can come from the group consisting of enzymes or hormones. Advantageously, it is also possible for the therapeutic active ingredient to come from the group consisting of antibodies or antibody fragments. These active ingredients are used as immunoglobulins for treating diseases. Also, it is possible to use antibodies or antibody fragments in the form of what are known as conjugates, and in this case a therapeutically effective molecule of the therapeutic active ingredient is coupled to an antibody or an antibody fragment.

It is also advantageous for the therapeutic active ingredient to be prepared as injectable liquid, as infusible liquid or as inhalant, even if the active ingredient is to be supplied later as, for example, a drug in tablet form or in another differing dosage form. Administration of the active ingredient by injection, infusion or inhalation leads, advantageously, to fast absorption, and so the distribution in the body can be determined comparatively quickly by PET after administration of the active ingredient. This is advantageous because the ¹¹C-labeled active ingredients have a short half life, and so, after administration of the active ingredient, there is only a short period of time available for detecting it in the body by PET.

The above-stated object is also achieved by a method for testing a therapeutic active ingredient, wherein the active ingredient already described above is used and the distribution of the active ingredient in the test subject is determined by positron emission tomography (PET). The advantages of this method, of providing additional information during testing of therapeutic active ingredients have already been explained. Alternatively, it is, advantageously, also possible to apply the therapeutic active ingredient to a tissue sample which actually represents the tissue to be treated. Here, too, it is possible to monitor the distribution of the therapeutic active ingredient by PET, and, advantageously, this method can be carried out without subjecting a human or animal test subject to stress.

It is advantageous when the distribution of the active ingredient in the test subject or in the tissue can be compared with a reference distribution in order to check the efficacy of the therapy with the active ingredient. The reference distribution can, for example, be values available from the literature which relate to the required concentration and distribution of certain active substance groups in the tissue to be treated. Thus, advantageously, it is possible to make better predictions of the effects of new therapeutic active ingredients if they are dependent on sufficient distribution in the tissue to be treated.

The reference distribution can also be determined by reference experiments in accordance with the method. For example, it is possible to establish a concentration of the therapeutic active ingredient in a tissue sample that is required in order to achieve the intended therapeutic outcome. Subsequently, in order to determine dosage guidelines for the therapeutic active ingredient, it is possible to study administration thereof in a human or animal test subject with the goal of achieving the required concentration of the active ingredient in the tissue to be treated of the test subject and of thus determining the required dosage of the drugs.

### BRIEF DESCRIPTION OF THE DRAWING

These and other objects and advantages of the present invention will become more apparent and more readily appreciated from the following description of the preferred embodiments, taken in conjunction with the accompanying drawing of which:

The single FIGURE shows an exemplary embodiment of the method proposed by the inventors.

### DETAILED DESCRIPTION OF THE PREFERRED EMBODIMENT

Reference will now be made in detail to the preferred embodiments of the present invention, examples of which are illustrated in the accompanying drawing, wherein like reference numerals refer to like elements throughout.

The therapeutic active ingredient with the radioactive ¹¹C-label is prepared in a system 11, which is represented diagrammatically by an essential element of the system, a cyclotron 11. After preparation of the active ingredient with label, it is applied close in time to a tissue sample 12 in a first phase of the testing and the distribution is checked by PET. This procedure can be repeated several times with modifications until a satisfactory result is achieved. Here, it is possible, for example, to generate a reference value which indicates the distribution the therapeutic active ingredient needs to be in to achieve the desired therapeutic effect in the tissue in question.

In a second phase, the radiolabeled therapeutic agent can be administered to a test animal 13. By PET, it is possible, for example, to determine the distribution of the therapeutic agent in the organ of the test animal 13, which contains the tissue to be treated 12. This means that, advantageously, initial statements can be made regarding how the therapeutic active ingredient is distributed in the living organism and whether the information obtained in the first phase can be applied.

In a third phase, the therapeutic active ingredient can then be administered to a human test subject 14. Here, too, the PET-determined values for the distribution can be compared with the reference value determined in the first phase. Also possible are statements with regard to whether the information obtained from phase II of the experiment is applicable to humans.

The preparation of, for example, ¹¹C-labeled amino acids is known per se. For example, J. Bolster et al. describe the labeling of tyrosine in the European Journal of Nuclear Medicine (1986), pages 321 to 324. This method can be used in the same way for the other amino acids present in peptides and proteins. In addition, the amino acids obtained in this way can be incorporated into peptides or proteins by customary methods. An α-¹¹C-labeled hydroxy acid can, for example, be synthesized by electrochemical reductive carboxylation of a ketone with incorporation of ¹¹CO_{2.}

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

1-9. (canceled)

10. A therapeutic active ingredient for administration to a human or animal body, the active ingredient containing at least one component selected from the group consisting of:
an α-hydroxy acid having a carboxyl group with an α-carbon atom,
an α-amino acid having a carboxyl group with an α-carbon atom,
a peptide having a peptide bond with a carbon atom, and
a protein having a peptide bond with a carbon atom,
wherein the active ingredient is radiolabeled with ¹¹C, such that the ¹¹C replaces the α-carbon atom of the α-hydroxy acid or of the α-amino acid, or replaces the carbon atom of the peptide bond of the peptide or of the protein.

11. The active ingredient as claimed in claim 10, wherein the active ingredient is an enzyme containing the component or a hormone containing the component.

12. The active ingredient as claimed in claim 10, wherein the active ingredient is an antibody containing the component or an antibody fragment containing the component.

13. The active ingredient as claimed in claim 10, wherein the active ingredient comprises:
an antibody containing the component or an antibody fragment containing the component; and
a therapeutically effective molecule coupled to the antibody or the antibody fragment.

14. The active ingredient as claimed in claim 10, wherein the active ingredient is an injectable liquid, an infusible liquid or an inhalant.

15. A method for testing a therapeutic active ingredient, to obtain information about a mode of action of the active ingredient, comprising:
administering the active ingredient to a test subject, the active ingredient containing at least one component selected from the group consisting of:
an α-hydroxy acid having a carboxyl group with an α-carbon atom,
an α-amino acid having a carboxyl group with an α-carbon atom,
a peptide having a peptide bond with a carbon atom, and
a protein having a peptide bond with a carbon atom,
wherein the active ingredient is radiolabeled with ¹¹C, such that the ¹¹C replaces the α-carbon atom of the α-hydroxy acid or of the α-amino acid, or replaces the carbon atom of the peptide bond of the peptide or of the protein; and
determining a distribution of the active ingredient in the test subject using positron emission tomography.

16. The method as claimed in claim 15, wherein the active ingredient is administered intravenously or by inhalation.

17. The method as claimed in claim 15, further comprising:
comparing the distribution of the active ingredient with a reference distribution to check efficacy of the active ingredient.

18. A method for testing a therapeutic active ingredient, comprising:
applying the active ingredient in vitro to a sample of a tissue to be treated with the active ingredient, the active ingredient containing at least one component selected from the group consisting of:
an α-hydroxy acid having a carboxyl group with an α-carbon atom,
an α-amino acid having a carboxyl group with an α-carbon atom,
a peptide having a peptide bond with a carbon atom, and
a protein having a peptide bond with a carbon atom,
wherein the active ingredient is radiolabeled with ¹¹C, such that the ¹¹C replaces the α-carbon atom of the α-hydroxy acid or of the α-amino acid, or replaces the carbon atom of the peptide bond of the peptide or of the protein; and
determining a distribution of the active ingredient in the sample of the tissue using positron emission tomography.

19. The method as claimed in claim 18, further comprising:
comparing the distribution of the active ingredient with a reference distribution to check efficacy of the active ingredient.

20. The method as claimed in claim 18, further comprising:
determining whether the active ingredient is therapeutically effective in the tissue; and
identifying an in vivo target concentration for the active ingredient based on the distribution of the active ingredient in the sample and whether the active ingredient was therapeutically effective.
